(19) United States
(12) Patent Application Publication
(10) Pub. No.: US 2005/0287225 A1
(43) Pub. Date: Dec. 29, 2005

(54) Metal cluster nano-compounds for treating tumor diseases

(76) Inventors: Gunter Schmid, Velbert (DE); Hubert Kuhn, Solingen (DE); Maria Tsoli, Essen (DE)

Correspondence Address: WOODARD, EMHARDT, MORIARTY, MCNETT & HENRY LLP, BANK ONE CENTER/TOWER, 111 MONUMENT CIRCLE, SUITE 3700, INDIANAPOLIS, IN, 46204-5137, US

(21) Appl. No.: 10/523,081

(22) PCT Filed: Jul. 31, 2003
(86) PCT No.: PCT/EP03/08475

§ 371 Date: Jul. 28, 2005

(30) Foreign Application Priority Data
Aug. 2, 2002 (DE) 10235602.5

(51) Int. Cl. A61K 33/24

(52) U.S. Cl. 424/617

(57) ABSTRACT

The invention relates to metal cluster nano-compounds of transition metals, which includes a metal nucleus formed from atoms of at least one transition metal and at least one ligand, and including their physiologically compatible salts, derivatives, isomers, hydrates, metabolites and prodrugs, which are suited for the prophylactic and/or therapeutic (curative) treatment of diseases of the human or animal body, particularly of tumor or cancer diseases. To this end, the inventive compounds can, under physiological conditions, interact with the DNA, preferably B-DNA, of the relevant cells.

The present invention relates to metal cluster nanocompounds, including their physiologically tolerated salts, derivatives, isomers, hydrates, metabolites and prodrugs, for the prophylactic and/or therapeutic (curative) treatment of disorders of the human and animal body, in particular of benign as well as malignant neoplastic and cancerous diseases.

The present invention relates in particular to the use of metal cluster nanocompounds, including their physiologically tolerated salts, derivatives, isomers, hydrates, metabolites and prodrugs, as pharmaceutical active compounds or drugs, in particular for preparing medicaments for the prophylactic and/or therapeutic (curative) treatment of neoplastic and cancerous diseases. The present invention equally relates to medicaments and pharmaceutical compositions which contain said metal cluster nanocompounds, including their physiologically tolerated salts, derivatives, isomers, hydrates, metabolites and prodrugs.

The present invention furthermore relates to a process for the prevention and/or treatment of disorders of the human or animal body, in particular of neoplastic and cancerous diseases, by using metal cluster nanocompounds, including their physiologically tolerated salts, derivatives, isomers, hydrates, metabolites and prodrugs.

Neoplastic and cancerous diseases do not represent a uniform condition but are generic terms for a multiplicity of various forms of benign as well as malignant disorders. Virtually any tissue of our body can produce cancerous degenerations, sometimes even a plurality of different types. Each of these conditions has in turn its own features. The causes for these disorders are often very heterogeneous.

Despite this diversity, virtually all tumors or cancerous degenerations are produced by very similar, fundamental molecular or cellular processes. In the last two decades, research has made astonishing progress in the knowledge concerning the most fundamental processes of cancerous or neoplastic events at the molecular level.

The DNA molecules of the chromosomes in the nucleus are the carriers of genetic information. Two classes of genes, which together form only a small proportion of the entire cellular makeup, play an essential role in the development of cancer, namely in particular proto-oncogenes (cancer gene precursors) and tumor suppressor genes (tumor-suppressing genes). They direct, in their normal form, the cellular life cycle and control the complicated sequence of processes which causes a cell to grow and, if necessary, to divide. Cell growth, while promoted by proto-oncogenes, is slowed down by tumor suppressor genes. These two classes of genes together are responsible for a large part of uncontrolled cell propagation processes in human tumors; if, for example, a proto-oncogene mutates in its regulatory or structural region, it may then happen that too much of its growth-promoting protein is produced or that said protein is excessively active; the proto-oncogene has then become a cancer-promoting oncogene which induces the cells to propagate excessively. In contrast, tumor suppressor genes contribute to the development of cancer when they are inactivated by mutations; as a result, the cell loses functional suppressor proteins and thus crucial growth inhibitors which normally prevent said cell from propagating disproportionally.

Normal somatic cells have a built-in emergency mechanism against unlimited propagation, which is a kind of counter which registers each cell division and which leads to a stop after a particular number of generations. After a particular, roughly predictable number of cell divisions or doublings, normal cells stop growing. This process is referred to cell ageing or senescence.

Responsible for this process of cell ageing or senescence at the molecular level are the DNA segments at the ends of the chromosomes, the “telomeres”. They register, as it were, how many propagation cycles a cell population undergoes and, from a particular point in time, induce senescence or crisis, thereby limiting the ability of a cell population to grow in an unrestricted manner.

In the case of most cancer or tumor cells, the above-described protective mechanism is no longer in force in the course of degeneration. It is therefore the aim of many therapeutic approaches to inhibit or to end growth or division of tumor or cancer cells, in particular to induce possibly blocking or even destruction of the tumor or cancer cell DNA. For this purpose, for example, platinum or ruthenium metal compounds, such as, for example, cis-diaminodichloroplatinum(II) (“cisplatin”), are used.

Interactions between metals and biological macromolecules, including proteins, polysaccharides and nucleic acids, are of particular interest, since they are crucially important to a multiplicity of natural and technical processes. These processes range from interactions between highly specific metal cofactors with particular proteins to biosorption of heavy metals by polysaccharide hydrogels.

The unique properties of DNA have resulted in the development of new materials, in particular in the field of medicine. However, conventional antitumor research is essentially focused on the interactions between platinum- and ruthenium-containing compounds with the major grooves and minor grooves of polynucleotides.

However, some of the previously used compounds have serious side effects. Thus, for example, cisplatin which binds to guanine of DNA and RNA is known to possess extreme nephrotoxicity which, in the worst case, can even result in necroses. There are furthermore a number of cisplatin-resistant tumors which are not accessible to a therapy with cisplatin.

It is thus the object of the present invention to find or provide active compounds and medicaments which are suitable in particular for the treatment of neoplastic or cancerous diseases, or else, where appropriate, of other disorders of the human or animal body.

Surprisingly, we have found that metal cluster nanocompounds of transition metals and physiologically tolerated salts, derivatives, isomers, hydrates, metabolites and prodrugs thereof are suitable for the prophylactic and/or therapeutic (curative) treatment of disorders of the human or animal body, in particular of neoplastic and cancerous diseases. These compounds can interact, under particular preconditions, with the DNA, in particular B-DNA, of human or animal cells, in particular of tumor or cancer cells, under physiological conditions.

The present invention thus relates to metal cluster nanocompounds of transition metals, which comprise a metal core of atoms of one or more transition metals and at least one ligand, and includes physiologically tolerated salts, derivatives, isomers, hydrates, metabolites and also prodrugs thereof for the prophylactic and/or therapeutic (curative) treatment of disorders of the human or animal body, with the average size of the metal core of said metal cluster metal cluster nanocompounds and/or the electronegativity of said metal cluster nanocompounds and/or the stabilization energy (i.e. the energy difference or potential difference between the free and the DNA-bound metal cluster nanocompound) being selected in a way so as to enable said metal cluster nanocompounds to interact with the DNA, preferably B-DNA, of human or animal cells, in particular of tumor or cancer cells, preferably under physiological conditions.

The term “metal cluster nanocompounds” refers, in accordance with the present invention, to compounds having metal-metal bonds—as opposed to the multinuclear complexes in the sense of Werner (see Römpp Chemielexikon, 10th Edition, Volume 1, 1996, Georg Thieme Verlag, pages 773/774, headword: “Cluster-Verbindungen” [Cluster compounds]). The term “cluster” or “cluster compounds” was introduced by F. A. Cotton in 1964.

The term “(metal) cluster” or “(metal) cluster compound” means, in accordance with the present invention, in particular a group or a core of 3 or more transition metal atoms each of which is chemically linked to at least 2 other atoms of the group or core, i.e. is at least part of a ring, with said group or core of transition metals being saturated or surrounded by suitable, in particular stabilizing ligands. The metal core of cluster compounds may consist of transition metal atoms of identical (mononuclear clusters) or different (heteronuclear clusters) transition metals. Such compounds contain ligands with a stabilizing action, examples of which are organic radicals, in particular those having free electron pairs (e.g. carbonyl radicals or triphenylphosphine radicals).

Thus, the term “(metal) cluster” or “(metal) cluster compound”, as used according to the invention, refers to the entire compound consisting of a metal core and ligands.

Thus, the metal cluster nanocompounds, as used according to the invention, are nanoparticles whose average diameter is in the range from a few Angstrom to a few nanometers and which consist of the actual metal core which is surrounded or saturated by ligands, in particular on its outer layer. Therefore it is also possible to use the term “metal nanocluster” synonymously for the term “metal cluster nanocompounds”.

Such metal cluster nanocompounds of transition metals are known per se from the prior art (see, for example, U.S. Pat. No. 5,521,289, U.S. Pat. No. B1-6,369,206 and U.S. Pat. No. 5,360,895). The use of such metal cluster nanocompounds for scientific purposes is also known already, for example the use of gold clusters for the imaging or microscopic viewability of DNA molecules (see, for example, Angew. Chem. 2002, 114, No. 13, pages 2429 to 2433, Willner et al. “Au-Nanoparticle Nanowires Based on DNA and Polylysine Templates”). However, no specific therapeutic application for these compounds has been described to date. This finding originates only from the inventors of the present application.

Possible examples of physiologically tolerated or acceptable salts of the metal cluster nanocompounds used according to the invention are salts of mineral acids, carboxylic acids or sulfonic acids; particular preference is given, for example, to salts of hydrochloric acid, hydrobromic acid, sulfuric acid, phosphoric acid, methanesulfonic acid, ethanesulfonic acid, toluenesulfonic acid, benzensulfonic acid, naphthalenedisulfonic acid, acetic acid, propionic acid, lactic acid, tartaric acid, citric acid, fumaric acid, maleic acid or benzoic acid. Examples of physiologically tolerated or acceptable salts which may be mentioned are also, however, salts containing conventional bases, such as, for example, alkali metal salts (e.g. sodium or potassium salts), alkaline earth salts (e.g. calcium or magnesium salts) or ammonium salts, derived from ammonia or organic amines such as, for example, diethylamine, triethylamine, ethyldiisopropylamine, procaine, dibenzylamine, N-methylmorpholine, dihydroabiethylamine, 1-ephenamine or methylpiperidine.

The present invention also comprises the derivatives of the metal cluster nanocompounds used according to the invention.

The present invention likewise comprises the isomers of the metal cluster nanocompounds used according to the invention. The term “isomers” is used, in accordance with the present invention, to include all possible isomeric forms. Nonlimiting examples of isomers which are also encompassed by the present invention are in particular stereoisomers, tautomers and constitutional isomers.

The present invention equally encompasses the hydrates of the metal cluster nanocompounds used according to the invention. According to the invention, “hydrates” refer to those forms of the metal cluster nanocompounds used according to the invention, which form a molecular compound (hydrate) with water by way of hydration in the solid or liquid state. In hydrates, the water molecules are complexed by intermolecular forces, in particular hydrogen bonds. Solid hydrates contain water as “crystal water” in stoichiometric or non-stoichiometric ratios, and the water molecules need not be equivalent, with respect to their binding state. Examples of hydrates are sesquihydrates, monohydrates, dihydrates, trihydrates etc. Equally suitable according to the invention are also the hydrates of salts.

Finally, the present invention also encompasses metabolites and prodrugs of the metal cluster nanocompounds used according to the invention. Metabolites refer, according to the invention, in particular to the metabolically produced or metabolically reacted products of the metal cluster nanocompounds used according to the invention. Prodrugs refer, according to the invention, in particular to those forms of the metal cluster nanocompounds used according to the invention, which themselves may be biologically active or inactive but which may be converted into the corresponding biologically active form (for example metabolically, solvolytically or in a different manner).

The metal cluster nanocompounds of transition metals, including their physiologically tolerated salts, derivatives, isomers, hydrates, metabolites and prodrugs, or the metal cores of such metal cluster nanocompounds may, as described above, interact, under particular preconditions, with the DNA, preferably B-DNA, of human or animal cells, in particular of tumor or cancer cells, under physiological conditions, for example by forming physical and/or chemical bonds. B-DNA is a special DNA conformity which can be found in aqueous media, in particular under physiological conditions, i.e. the hydrated form

In order to be able to interact with the DNA, preferably B-DNA, of human or animals cells, in particular of tumor or cancer cells, the average size of the metal core of the metal cluster nanocompounds and/or the electronegativity of said metal cluster nanocompounds and/or the stabilization energy (i.e. the energy difference or potential difference between the free and the DNA-bound metal cluster nanocompound) must be selected so as to make possible such an interaction.

Regarding the size of the metal core of the metal cluster nanocompounds, the selection should be carried out so as for the average size of the metal cores of the metal cluster nanocompounds to be such that they are able to attach to the major grooves of the DNA molecules, in particular of B-DNA, of the tumor or cancer cells.

For this purpose, the average size of the metal cores of the metal cluster nanocompounds should be no more than about 2.5 nm, in particular no more than about 2.0 nm, preferably no more than about 1.6 nm, particularly preferably no more than about 1.5 nm, very particularly preferably about 1.4 nm and at least about 0.5 nm, in particular at least about 0.75 nm, preferably at least about 1.0 nm, particularly preferably at least about 1.3 nm. Particular preference is given to the average size of the metal cores of the metal cluster nanocompounds being in the range from about 1.3 nm to about 1.5 nm.

With regard to the stabilization energy, the metal cluster nanocompounds used according to the invention should be selected so as for the stabilization energy _E^{stab} of the interaction(s), in particular bond(s), between said metal cluster nanocompound (MCN) and the DNA, in particular B-DNA, calculated as a potential difference between, on the one hand, the sum of the potential energies of the ligand-free metal core of said metal cluster nanocompound, E^{pot}_{MCN}, and the free DNA, E^{pot}_{DNA}, and, on the other hand, the potential energy of the resulting complex of the ligand-free metal core of the metal cluster nanocompound and DNA, E^{pot}_{MCN-DNA}:
ΔE^{stab}=(E^{pot}_{MCN}+E^{pot}_{DNA})−E^{pot}_{MCN-DNA}
is, under normal conditions, at least about −400 kJ/mol, in particular at least about −625 kJ/mol, preferably at least about −825 kJ/mol, particularly preferably at least about −1000 kJ/mol, very particularly preferably about −1200 kJ/mol. Normal conditions mean, in the present case, in particular a temperature in the range from 0 to 50° C., in particular about (20±5)° C., and a pressure in the range from 10⁴ to 10⁶ Pa, in particular about 1.01325·10⁵ Pa.

In this context, the value indicated for the stabilization energy ΔE^{stab} refers to the reaction of a ligand-free metal core with a DNA molecule. E^{pot}_{MCN} denotes the potential energy of a ligand-free metal core of the metal cluster nanocompound, i.e. of a “naked” metal core in the (ligand) free state, i.e. prior to attachment to the DNA molecule. E^{pot}_{DNA} denotes the potential energy of a free DNA molecule, in particular B-DNA, i.e. before the interaction with or binding to the metal core of the metal cluster nanocompound occurs. E^{pot}_{MCN-DNA} denotes the potential energy of the product or complex of the reaction of the one ligand-free metal core with the one DNA molecule, in particular in the B conformation.

As illustrated before, the selection with respect to the electronegativity of the metal cluster nanocompounds used according to the invention must be carried out in such a way that said metal cluster nanocompounds can interact with the DNA, in particular B-DNA, of tumor or cancer cells. A suitable measure of the electronegativity of the particular metal cluster nanocompound may be the redox potential E° of the transition metal forming the metal core of the metal cluster nanocompound in the electrochemical series. In the metal cluster nanocompounds useable according to the invention, the redox potential, i.e. the normal potential E°, of the transition metal forming the metal core should be greater than 0 V, in particular greater than +0.25 V, preferentially greater than +0.5 V, preferably greater than +0.75 V, particularly preferably greater than +1.0° V, in each case based on the redox potential of the normal hydrogen electrode of 0 V (zero point). Preference is given to platinum (E°=+1.20 V) and gold (E°=+1.50 V) as transition metals forming the metal core of the particular metal cluster nanocompound, and particular preference is given to gold as the most electropositive of all metals. For further details regarding the electrochemical series and redox potentials, reference may be made to Römpp Chemielexikon, 10th Edition, Volume 5, 1998, Georg Thieme Verlag, pages 4162/4163, headword: “Spannungsreihe” [Electrochemical series].

According to a preferred embodiment, the metal core of the metal cluster nanocompounds used according to the invention contains at least 30 metal atoms, in particular at least 40 metal atoms, preferably at least 50 metal atoms, particularly preferably at least 55 metal atoms and, respectively, no more than 90 metal atoms, in particular no more than 80 metal atoms, preferably no more than 70 metal atoms, particularly preferably no more than 60 metal atoms. Preference is given according to the invention to metal cores having from 50 to 70 metal atoms.

In the metal cluster nanocompounds preferably used according to the invention, the transition metal of the metal core is selected from the group consisting of platinum (Pt), gold (Au), rhodium (Rh), iridium (Ir), palladium (Pd), ruthenium (Ru), osmium (Os) and silver (Ag) and also mixtures thereof, preferably from the group consisting of platinum (Pt), gold (Au) and ruthenium (Ru) and mixtures thereof. Particular preference is given to gold (Au).

In order to achieve good physiological efficacy and applicability, the metal cluster nanocompounds should be selected so as to be soluble or at least dispersible in aqueous media, in particular under physiological conditions. This may be controlled, in particular, by selecting suitable ligands.

Examples of ligands suitable according to the invention are organic radicals or halogens, preferably chlorine. Examples of organic compounds suitable according to the invention are, for example, triphenylphosphine and its derivatives, in particular sulfonated derivatives (e.g. P(C₆H₅)₂(C₆H₄SO₂H)).

Metal cluster nanocompounds preferred according to the invention have a metal core which comprises from 50 to 70 metal atoms, preferably 55 metal atoms, and which has an average size of from about 0.5 nm to about 2.5 nm, in particular from about 1.0 nm to about 1.5 nm. In this connection, the metal core, including ligand(s), may in particular have average sizes of from 1 to 5 nm, in particular 2 to 3 nm, preferably about 2.5 nm. Metal cluster nanocompounds which are particularly preferred according to the invention have an Au₅₅ metal core which is surrounded by one or more suitable ligands.

According to a particular embodiment of the present invention, metal cluster nanocompounds of the general formula (I)
[MₙLₘ] (I)
including their physiologically tolerated salts, derivatives, isomers, hydrates, metabolites and/or prodrugs are used, in which formula (I):
M is a transition metal atom which may be selected preferably from the group consisting of platinum (Pt), gold (Au), rhodium (Rh), iridium (Ir), palladium (Pd), ruthenium (Ru), osmium (Os) and silver (Ag) and also mixtures thereof, particularly preferably from the group consisting of platinum (Pt), gold (Au) and ruthenium (Ru) and also mixtures thereof and which is very particularly preferably gold (Au), it being possible for M to denote identical or different metals in the same metal cluster nanocompound;
n is the number of transition metal atoms per metal cluster nanocompound, with n being at least 30, in particular at least 40, preferably at least 50, particularly preferably at least 55, and no higher than 90, in particular no higher than 80, preferably no higher than 70, particularly preferably no higher than 60, and, very particularly preferably, is in the range from 50 to 70;
L is a ligand, in particular an organic radical, and may denote identical or different ligands in the same molecule;
m is the number of ligands per molecule and is at least 10, in particular at least 12, preferably at least 18.

In the above formula (I), preference is given M=Au and/or n=55. The ligand L in the above formula (I) is preferably selected from the group consisting of triphenylphosphine and its derivatives, in particular sulfonated derivatives; halogens, in particular chlorine; and mixtures thereof.

According to a particular embodiment of the present invention, metal cluster nanocompounds of the general formula (II)
[Au₅₅L′₁₂X₆] (II)
are used, in which formula (II)
L′ is a ligand, in particular an organic radical, where L′ may denote identical or different ligands in the same molecule and L′ is in particular a triphenylphosphine radical or derivatives thereof, in particular sulfonated derivatives, particularly preferably P(C₆H₅)₂(C₆H₄SO₃H), very particularly preferably P(C₆H₅)₂ (meta-C₆H₄SO₃H);
X is a halogen atom, preferably chlorine, and may denote identical or different halogen atoms in the same molecule.

Preference is given according to the invention to metal cluster nanocompounds of the formula
[Au₅₅{P(C₆H₅)₂(C₆H₄SO₃H)}₁₂Cl₆]
including their physiologically tolerated salts, derivatives, isomers, hydrates, metabolites and/or prodrugs.

According to the invention, particular preference is given to the metal cluster nanocompound of the formula
[Au₅₅{P(C₆H₅)₂(meta-C₆H₅SO₃H)}₁₂Cl₆]
including its physiologically tolerated salts, derivatives, isomers, hydrates, metabolites and/or prodrugs.

The compounds [Au₅₅{P(C₆H₅)₂(C₆H₄SO₃H)}₁₂Cl₆] and [Au₅₅{P(C₆H₅)₂(meta-C₆H₄SO₃H)}₁₂Cl₆] may be prepared by means of ion exchange of the corresponding sodium sulfonates, [Au₅₅P(C₆H₅)₂(C₆H₄SO₃Na)}₁₂Cl₆] and [Au₅₅{P(C₆H₅)₂ (meta-C₆H₄SO₃Na)}₁₂Cl₆], respectively, on acidic ion exchangers (Angew. Chem. Int. Ed. Eng. 1995, 34, No. 13/14, pages 1442 ff., G. Schmid et al. “First Steps Towards Ordered Monolayers of Ligand-Stabilized Gold Clusters”). The sodium sulfonates are in turn obtained by the following phase transfer reaction (Polyhedron, Vol. 7, No. 22/23, 1988, pages 2321 to 2329, G. Schmid “Metal Clusters And Cluster Metals”):

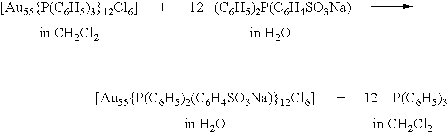

Finally, it is possible to prepare the compound [Au₅₅{P(C₆H₅)₃}₁₂Cl₆] by reacting [AuCl{P(C₆H₅₎₃}] with diborane, B₂H₆, in warm benzene or toluene (Inorganic Syntheses, Vol. 27, Edition A. P. Ginsberg, John Wiley 1990, Section 41 “Hexachlorododecakis(triphenylphosphine)pentapentacontagold”, pages 214 to 218). The corresponding rhodium, ruthenium and palladium complexes can be prepared in a similar manner (see Inorganic Syntheses, Vol. 27, Edition A. P. Ginsberg, John Wiley 1990 and references cited therein).

In order to ensure particularly good applicability of the metal cluster nanocompounds described above, in particular also under physiological conditions, the metal cluster nanocompounds of the type described above, used and selected according to the invention, advantageously have good water solubility, in particular a water solubility of at least 0.1 μmol/l, preferably at least 1.0 μmol/l, particularly preferably at least 1 mmol/l or more and up to 100 mmol/l and more.

The metal cluster nanocompounds described above, including their physiologically tolerated salts, derivatives, isomers, hydrates, metabolites and prodrugs, possess a previously unrecognized therapeutic potential with respect to the treatment of disorders of the human or animal body, in particular of neoplastic and/or cancerous diseases, including the treatment of primary tumors, metastases and precancerous conditions (pre-cancer stages). Thus, the above-described metal cluster nanocompounds are suitable for the prophylactic and therapeutic or curative treatment of benign as well as malignant tumors, in particular, for example, for the treatment of colon cancer (colon carcinomas), breast cancer (mamma carcinomas), ovarian carcinomas, carcinomas of the uterus, lung cancer, stomach cancer, liver cancer, carcinomas of the pancreas, kidney cancer, bladder cancer, prostate cancer, testicular cancer, bone cancer, skin cancer, Kaposi sarcomas, brain tumors, myosarcomas, neuroblastomas, lymphomas and leukemias.

The aforedescribed metal cluster nanocompounds used according to the invention, including their physiologically tolerated salts, derivatives, isomers, hydrates, metabolites and prodrugs, were found to be capable of inhibiting or halting the growth and division of tumor and cancer cells, even of inducing destruction of the tumor- and cancer-cell DNA.

Thus the aforedescribed metal cluster nanocompounds used according to the invention were found to be particularly effective in in-vitro studies, even on cisplatin-resistant tumors. In comparison with cisplatin, a distinctly improved efficacy was found in the treatment of tumors which are not resistant to cisplatin.

It is assumed, without being committed to a particular theory, that the metal cluster nanocompounds used according to the invention are deposited in the major grooves of the DNA, in particular B-DNA, of tumor or cancer cells and are capable of interacting there with said DNA.

Compounds having an Au₅₅ core, in particular the compounds [Au₅₅{P(C₆H₅)₂(C₆H₄SO₃H)}₁₂Cl₆] and [Au₅₅{P(C₆H₅)₂(meta-C₆H₄SO₃H)}₁₂Cl₆], have been found to be particularly effective in this context. Studies by the applicant have found that the free acid has an even stronger pharmaceutical potential or superior efficacy in comparison with the corresponding alkali sulfonate. Without being committed to a particular theory, the efficacy of these compounds can possibly be explained by the fact that they interact with the GCA base sequences of the DNA in question.

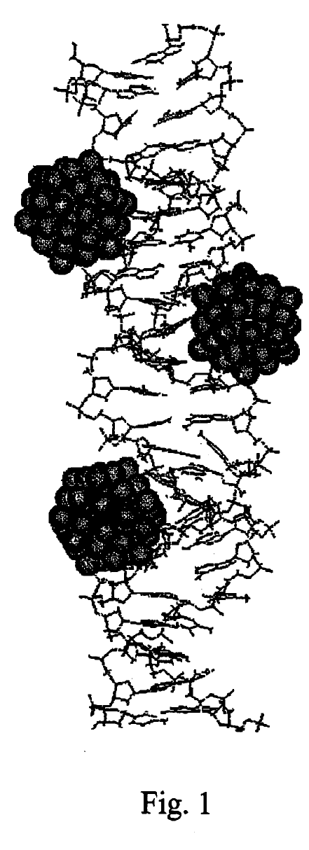

FIG. 1 depicts diagrammatically the incorporation of three metal cores of metal cluster nanocompounds used according to the invention, in particular Au₅₅-cores, into the major grooves of a B-DNA strand of a cancer or tumor cell, with the ligands not being depicted in the diagrammatic illustration. In this way, the Au₅₅ cores which have been arranged in the major grooves of the DNA and which have interacted with the latter then prevent the DNA from dividing and thus propagation of the corresponding cell

The present invention further relates to the use of the aforedescribed metal cluster nanocompounds, including their physiologically tolerated salts, derivatives, isomers, hydrates, metabolites and prodrugs, as pharmaceutical active compounds (drugs), together with a pharmaceutically tolerated, essentially nontoxic carrier or excipient.

The present invention further relates to pharmaceutical compositions or medicaments which comprise at least one metal cluster nanocompound as described above or its physiologically tolerated salts, derivatives, isomers, hydrates, metabolites and/or prodrugs together with a pharmaceutically tolerated, essentially nontoxic carrier or excipient.

The present invention further relates to a process for the prevention or treatment of disorders of the human or animal body, in particular of neoplastic and cancerous diseases, as defined above, by using at least one metal cluster nanocompound as described above and/or its physiologically tolerated salts, derivatives, isomers, hydrates, metabolites and/or prodrugs in therapeutically active amounts together with a pharmaceutically tolerated, essentially nontoxic carrier or excipient.

The metal cluster nanocompounds used according to the invention or their physiologically tolerated salts, derivatives, isomers, hydrates, metabolites and prodrugs may, where appropriate, be used in combination with a further pharmaceutical active compound, in particular a chemotherapeutic and/or a cytostatic agent, either as a functional unit, in particular in the form of a blend, a mixture or a batch, or else (spatially) separated from one another.

The active compounds or active compound combinations used according to the invention may be administered systematically or else topically, in particular locally, depending on the type of the disorders to be treated.

Any customary forms of administration are suitable for administering the active compounds or active compound combinations used according to the invention. Administration may be carried out, for example, orally, lingually, sublingually, buccally, rectally or parenterally (i.e. by circumventing the intestinal tract, i.e. intravenously, intraarterially, intracardially, intracutaneously, subcutaneously, transdermally, intraperitoneally or intramuscularly), with oral and intravenous administration being particularly suitable; very particular preference is given to oral administration. A topical application is also possible (e.g. for the treatment of melanomas).

A particular form of topical application consists of introducing the active compounds or active compound combinations into a carrier system, in particular a drug delivery system, and implanting said carrier system into the neoplastic or cancerous tissue or at least close to or in the environment of said neoplastic or cancerous tissue, where said carrier system then releases said active compounds or active compound combinations specifically at the site of said neoplastic or cancerous tissue. In this way it is possible to avoid side effects, as may occur in the case of systemic administration, i.e. to reduce the overall strain on the body markedly. Examples of implantable carrier or drug delivery systems suitable according to the invention are described in the international laid-open publication WO 00/25841 A1, which originates from the applicant herself and whose entire contents are hereby incorporated by reference. The carrier or drug delivery system described in WO 00/25841 A1 enables, for example, the release of active compounds or active compound combinations to be specifically controlled (for example by varying the size of the openings for releasing said active compounds or active compound combinations, by chemical modification of the surface, etc.).

For application according to the invention, the active compounds or active compound combinations are transferred into the usual formulations such as, for example, tablets, sugar-coated tablets, pills, granules, aerosols, syrups, emulsions, suspensions, solutions, ointments, creams and gels of any kind, in particular by using inert, essentially nontoxic, pharmaceutically suitable carriers or solvents. To this end, the active compounds or active compound combinations used according to the invention may be present in each case at a therapeutically active concentration, in particular at concentrations of from about 0.0001 to about 99% by weight, preferably from about 0.01 to about 95% by weight, of the total mixture, i.e. in amounts sufficient to achieve the indicated or desired dosage range. Nevertheless, it may be necessary, where appropriate, to deviate from the abovementioned amounts, namely depending on the body weight or on the type of route of administration, on the individual reaction to the medicament, on the type of formulation and on the time or interval of administration. Thus it may be sufficient, in some cases, to manage with less than the aforementioned minimal amount, while in other cases the upper limit mentioned has to be exceeded. In the case of administering relatively large amounts, it may be recommended to distribute said amounts in the form of several single doses over a defined period of time, for example during the day.

The formulations are prepared, for example, by diluting the active compounds or active compound combinations with solvents (e.g. oils such as castor oil) and/or carriers, where appropriate by using emulsifiers and/or dispersants, it being possible, for example in the case of utilizing water as a diluent, to use, where appropriate, organic solvents as auxiliary solvents.

Depending on the type of administration, it has proved advantageous to administer the active compounds or active compound combinations used according to the invention in amounts of from about 0.0001 to about 500 mg/kg of body weight, in particular from about 0.0001 to about 100 mg/kg, preferably 0.01 to 50 mg/kg, in order to achieve more effective results. Nevertheless, it may be necessary, where appropriate, to deviate from the abovementioned amounts, namely depending on the body weight or on the type of route of administration, on the individual reaction to the medicament, on the type of formulation and on the time or interval of administration. Thus it may be sufficient, in some cases, to manage with less than the aforementioned minimal amount, while in other cases the upper limit mentioned has to be exceeded. In the case of administering relatively large amounts, it may be recommended to distribute said amounts over a defined period of time, for example during the day, that is, for example, in the form of several single doses or of continuous administration (e.g. continuous infusion). The application in a chronic therapy (e.g. in tablet form) is likewise possible.

Further embodiments, modifications and variations of the present invention are immediately obvious to the skilled worker by reading the present specification and can be implemented by him without leaving the scope of the present invention.

The present invention is illustrated on the basis of the following exemplary embodiments which, however, do not limit the present invention in any way.

### EXAMPLE EMBODIMENTS

### Example 1

### A): Preparation of [Au₅₅{P(C₆H₅)₃}₁₂Cl₆]

The compound [Au₅₅{P(C₆H₅)₃}₁₂Cl₆] is prepared according to Inorganic Syntheses, Vol. 27, Edition A. P. Ginsberg, John Wiley 1990, Protocol No. 41, pages 214 to 218. For this, AuCl[P(C₆H₅)₃] is reacted with diborane, B₂H₆, in warm benzene or toluene.

The diborane, B₂H₆, itself can be prepared according to the following equation:
3NaBH₄+4BF₃.O(C₂H₅)₂→3NaBF₄+2B₂H₆+4(C₂H₅)₂O

The compound [Au₅₅{P(C₆H₅)₃}₁₂Cl₆] is a dark-brown powder which can be dissolved in dichloromethane and pyridine.

### Example 1

### B): Preparation of [Au₅₅{P(C₆H₅)₂(C₆H₄SO₃Na)}₁₂Cl₆]

[Au₅₅{P(C₆H₅)₂(C₆H₄SO₃Na)}₁₂C₆] is prepared by reacting the compound prepared in example 1. A) with [P(C₆H₅)₃] in a phase transfer reaction according to the following equation (see Polyhedron, Vol. 7, No. 22/23, 1988, pages 2321 to 2329):

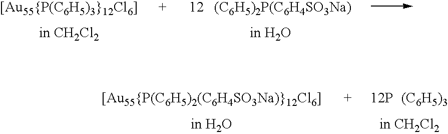

### Example 1

### C): Preparation of [Au₅₅{P(C₆H₅)₂(C₆H₄SO₃H)}₁₂Cl₆]

The free sulfonic acid, [Au₅₅{P(C₆H₅)₂(C₆H₄SO₃H)}₁₂Cl₆], can be prepared starting from the Au₅₅-cluster compound prepared in example 1. B), by applying the latter to an acidic ion exchanger (Angew. Chem. Int. Ed. Engl. 1995, 34, No. 13/14, pages 1442 ff). The free acid proves to be particularly effective, with respect to tumor or cancer cells, in the in-vitro cell toxicity measurements described below.

### Example 2

### In-Vitro Cell Toxicity Measurements

The in-vitro cytotoxicity properties of the gold-55 particles (Au₅₅) prepared in example 1. C) were carried out on HeLa cancer cells and on MOR/P and MOR/CPR lung cancer cells. MOR/P cells are sensitive to cisplatin, while MOR/CPR cells are resistant to cisplatin.

The HeLa cells were grown on a DMEM medium at 37° C. in a 5% CO₂ atmosphere. The medium had been supplemented with 10% strength FCS serum and antibiotics. Daughter cultures were generated twice weekly. The MOR/P and MOR/CPR cells were grown on an RPMI 1640 medium at 37° C. in a 5% CO₂ atmosphere. Said medium had likewise been supplemented with 10% strength FCS serum and antibiotics. Here too, daughter cultures were generated twice weekly.

The in-vitro cytotoxicity of the Au₅₅ particles was determined in the following manner using 96-well microtiter plates and an MTT colorimetry assay (CellTiter 96® Aqueous One Solution Cell Proliferation Assay, Promega):

Cultures of each cell line were applied at a concentration of 1_{—}10⁵ cells/ml to the microtiter plates and grown in the above-described media at 37° C. in a 5% CO₂ atmosphere for 72 hours. Au₅₅ particles were then dissolved in each case in 50 μl of the RPMI and DMEM media and added in such a way so as to produce the following Au₅₅ concentrations: 0.5, 0.75, 1.0, 3.0, 6.0 10.0 and 50.0 μm. A reaction mixture of control cells without gold-55 particles contained 50 μl of the DMEM or RPMI medium. The microtiter plates were incubated at 37° C. in a 5% CO₂ atmosphere for 15 hours.

After incubating the cancer cells with Au₅₅ particles, 40 μl of the MTT reagent were added to each well of each microtiter plate. This was followed by 4 hours of incubation at 37° C.

Absorption at 490 nm was measured for each well of each microtiter plate by using a 96-well microtiter plate reader. The absorption measured at 490 nm was plotted as a function of the Au₅₅ particle concentration and the IC₅₀ value was determined.

Diagram 1 depicts the profile of the sensitivity of HeLa cancer cells to the Au₅₅ particles prepared in example 1. C). The graph shows the absorption profile at 490 nm after incubation as a function of the increase in Au₅₅ particle concentration. The experiment was confirmed by repeating it three times. The IC₅₀ value (50% of cells are inactive) for this cell line was determined at an Au₅₅ concentration of 5.0 μM. To date, nothing is known about the IC₅₀ value for cisplatin and HeLa cancer cells.

Diagram 2 depicts the profile of the sensitivity of cisplatin-sensitive human MOR/P lung cancer cells to the Au₅₅ particles prepared in example 1. C). The graph depicts the absorption profile at 490 nm after incubation and comparison with the control cells as a function of the increase in Au₅₅ particle concentration. The individual lines indicate independent experiments each of which was repeated three times. The IC₅₀ value for this cell line was determined at an Au₅₅ concentration of 2.1±0.07 μM. The IC₅₀ value for this cell line for cisplatin is 3.3±0.3 μM.

Diagram 3 depicts the profile of the sensitivity of cisplatin resistant human MOR/CPR lung cancer cells to the Au₅₅ particles prepared in example 1. C). The graph shows the absorption profile at 490 nm after incubation and comparison with the control cells as a function of the increase in Au₅₅ particle concentration. The individual lines indicate independent experiments each of which was repeated three times. The IC₅₀ value for this cell line was determined at an Au₅₅ concentration of (2.0±0.21) μM. The IC₅₀ value for this cell line for cisplatin is (7.1±1.2) μM.

### Example 3

### DNA Cleavage by Restriction Endonucleases in the Presence of the Au₅₅ Particles Prepared in Example 1. C)

Restriction enzymes are known to cleave double-strand deoxyribonucleases at specific base sequences. Restriction endonucleases were used for DNA cleavage in order to investigate whether the Au₅₅ particles interact preferably with specific nucleotides (bases).

To this end, the following enzymes were used: Sma I (Roche), Hind III (Gibco), Pst I (Gibco) and Sal I (Gibco). These enzymes cleave DNA at in each case different sites (base sequences).

DNA cleavage by the various restriction endonucleases was determined in a 30 μl volume by the following process: after preincubation at room temperature for 15 hours, the Au₅₅ particles were added to 0.1 μg/μl plasmid DNA (pcDNA3.1/myc-His© (−) B, Invitrogen), with a final Au₅₅ particle concentration of 5 μM. Subsequently, the particular enzyme (20 units/μl) and 6 μl of an appropriate enzyme buffer solution were added.

The DNA was cleaved, in the case of Hind III, Pst I and Sal I, at 37° C. and, in the case of Sma I, at 25° C. for two hours. The cleaving process was stopped by way of heat inactivation, i.e. by incubating the reaction solution at a temperature of 65° C. for 20 minutes and subsequently at −20° C. for 10 minutes. The cleaved DNA was made visible via gel electrophoresis. The results of the experiments are summarized in table 1 below:

TABLE 1
Au₅₅-treated DNA cleavage by restriction
enzymes

| | Sma I | Hind III | Pst I | Sal I |
|---|---|---|---|---|
| | CCC ↓ GGG | A ↓ AGCTT | CTGCA ↓ G | G ↓ TCGAC |
| Au₅₅ | +/−50% | +/−50% | +/−90% | +/−50% |
| 5 M | | | | |

Deactivation is indicated by +/− and a corresponding percentage. It is obvious that the Au₅₅ particles mainly inhibit the Pst I restriction enzyme in cleaving the CTGCAG base sequence. Thus it can be concluded that the Au₅₅ particles preferably interact with the GCA base sequence.

### Example 4

### Investigation of the Antitumor Potential of the Au₅₅-Cluster Compound Au₅₅(Ph₂PC₆H₄SO₃H)₁₂Cl₆ (with Ph=Phenyl)

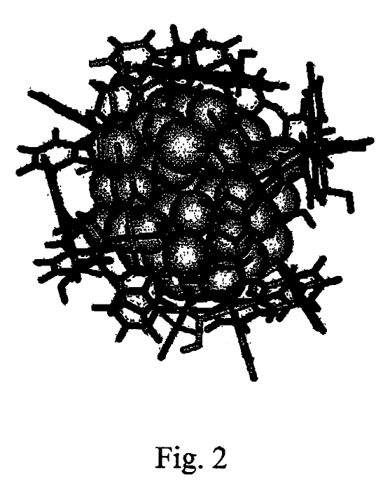

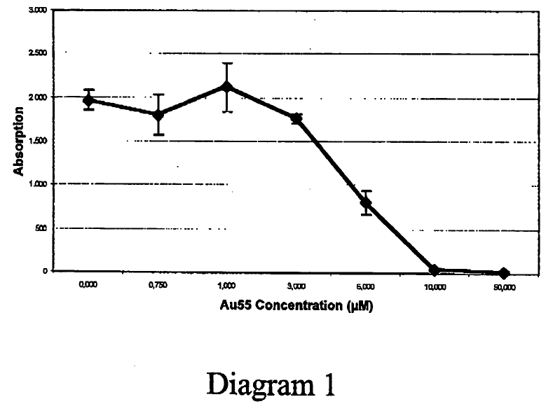

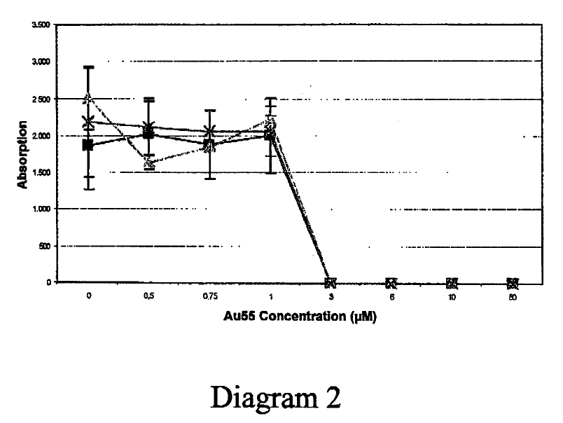

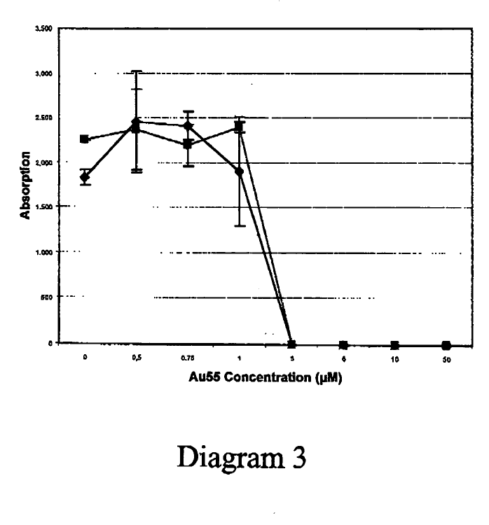

In the present exemplary embodiment, the antitumor potential of the compound Au₅₅(Ph₂PC₆H₄SO₃H)₁₂Cl₆, sometimes referred to only as [Au₅₅] hereinbelow, for a number of human cancer cell lines was investigated. The Au₅₅ clusters consist, in addition to a core of 55 gold atoms, of a shell of 12 water-soluble, monosulfonated triphenylphosphane molecules and 6 chlorine atoms (FIG. 2 which depicts the model of an Au₅₅(PPh₃)₁₂Cl₆ cluster).

The in-vitro cytotoxicity was studied by means of the MTT assay (Promega), a colorimetric method in which a tetrazolium-based compound is reduced by living cells to give formazan. The amount of formazan formed is directly proportional to the number of living cells in the culture. Each cell line was incubated in microliter dishes for 48 hours before adding the medicaments. Cisplatin or Au₅₅ was added, followed by an incubation for 72 or 24 hours. Subsequently, the MTT assays were carried out following Promega's information.

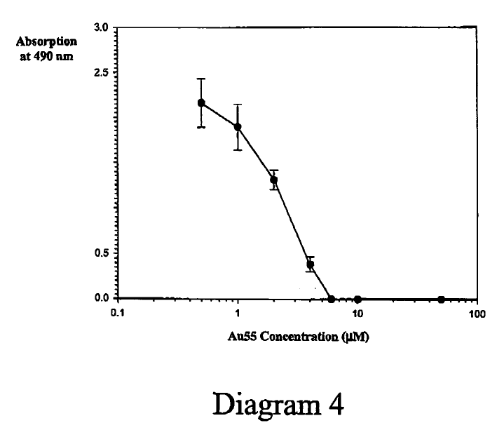

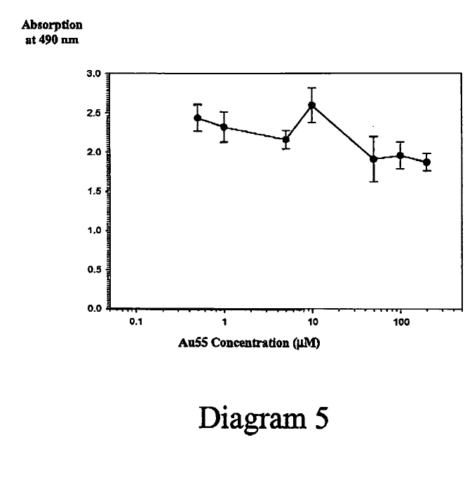

Diagram 4 depicts a typical graph of an MTT assay. Absorption by formazan and thus the life span of the cells decreases with increasing [Au₅₅] concentration. It was likewise investigated whether the ligand molecules themselves influence the life span of the cells. This cannot be detected in the case of the MOR/CPR tumor cell line studied (FIG. 5). In detail:

Diagram 4 relates to in-vitro cytotoxicity assays on cisplatin-resistant MOR/CPR lung tumor cells, incubated with different concentrations of Au₅₅(Ph₂PC₆H₄SO₃H)₁₂Cl₆ [Au₅₅] for 24 hours. Each point represents 3 experiments carried out independently of one another and repeated in each case three times.

Diagramm 5 relates to in-vitro cytotoxicity assays on cisplatin-resistant MOR/CPR lung tumor cells incubated with different concentrations of free ligand, Ph₂PC₆H₄SO₃H, for 24 hours. Each point represents 3 independent experiments repeated in each case in triplicate.

For the cell lines studied, [Au₅₅] was generally found to have faster and higher cytotoxicity than cisplatin, as is apparent from the IC₅₀ data in table 2. The only previously tested healthy cells, namely those of MC3, characteristically respond more weakly to [Au₅₅] than the bone tumor cells U20S. This leads to the conclusion that [Au₅₅] is less toxic to healthy cells than to tumor cells. Experiments with healthy skin cells and tumor skin cells (melanoma) show the same tendency. Also remarkable is the fact that metastatic melanoma cells are resistant to cisplatin but extremely sensitive to [Au₅₅]. Thus there is the possibility of applying [Au₅₅] particularly in cases in which resistance to cisplatin occurs.

Table 2 below depicts the inhibitory concentrations of cisplatin and [Au₅₅] incubations with various cell lines over 72 and 24 hours, respectively. The IC₅₀ data were calculated from the graphs obtained from the in vitro cytotoxicity assays MTT. Each experiment was repeated three times independently from one another by way of determination in triplicate.

TABLE 2

| Cell | | IC₅₀ cisplatin | | IC₅₀ [Au₅₅] |
|---|---|---|---|---|
| line | | 72 h | | 24 h |
| MC3 | Normal bone cells | 26.1 ± 1.27 | μM | 1.65 ± 0.14 μM |
| U205 | Osteosarcoma | 11.17 ± 2.02 | μM | 0.64 ± 0.04 μM |
| MOR/P | Lung cancer cells, | 3.30 ± 0.3 | μM | 2.10 ± 0.10 μM |
| | cisplatin-sensitive | | | |
| MOR/CPR | Lung cancer cells, | 7.10 ± 1.2 | μM | 2.50 ± 0.10 μM |
| | cisplatin-resistant | | | |
| BLM | Metastatic melanoma | 54.70 ± 7.60 | μM | 0.30 ± 0.10 μM |
| MV3 | Metastatic melanoma | >50 | μM | 0.24 ± 0.02 μM |
| HeLa | Cervical cancer | 7.93 ± 0.95 | μM | 2.29 ± 0.10 μM |
| | cells | | | |
| Hek | Kidney cancer cells, | 20.13 ± 6.0 | μM | 0.63 ± 0.02 μM |
| | transfected with | | | |
| | adenovirus | | | |

1-36. (canceled)

37. A metal cluster nanocompound of transition metals, comprising a metal core and at least one ligand, and physiologically tolerated salts, derivatives, isomers, hydrates, metabolites and prodrugs thereof,
wherein at least one of: the average size of the metal core of said metal cluster nanocompounds, the electronegativity of said metal cluster nanocompounds, and the stabilization energy ΔE^{stab} are being selected to enable said metal cluster nanocompounds to interact with the DNA under physiological conditions, for the prophylactic and/or therapeutic treatment of disorders of the human or animal body.

38. The metal cluster nanocompound as claimed in claim 37, in which the interaction between said metal cluster nanocompound and the DNA takes place by way of physical and/or chemical bond(s) and/or interaction(s).

39. The metal cluster nanocompound as claimed in claim 37, in which the stabilization energy ΔE^{stab} of the interaction(s) between said metal cluster nanocompound (MCN) and the DNA calculated as a potential difference between, on the one hand, the sum of the potential energies of the ligand-free metal core of said metal cluster nanocompound, E^{pot}_{MCN}, and the free DNA, E^{pot}_{DNA}, and, on the other hand, the potential energy of the resulting complex of the ligand-free metal core of the metal cluster nanocompound and DNA, E^{pot}_{MCN-DNA}:
ΔE^{stab}=(E^{pot}_{MCN}+E^{pot}_{DNA})−E^{pot}_{MCN-DNA}
is at least about −400 kJ/mol.

40. The metal cluster nanocompound as claimed in claim 37, in which the average size of the metal core of said metal cluster nanocompounds is selected in a way so as to enable said nanocompounds to attach to the major grooves of the DNA molecules.

41. The metal cluster nanocompound as claimed in claim 37, in which the metal cores of said metal cluster nanocompounds have an average size of from about 0.5 nm to about 2.5 nm.

42. The metal cluster nanocompound as claimed in claim 37, in which the metal core of said metal cluster nanocompounds has at least 30 metal atoms and no more than 90 metal atoms.

43. The metal cluster nanocompound as claimed in claim 37, in which the transition metals are selected from the group consisting of: platinum (Pt), gold (Au), rhodium (Rh), iridium (Ir), palladium (Pd), ruthenium (Ru), osmium (Os), silver (Ag), and mixtures thereof.

44. The metal cluster nanocompound of claim 37, in which the metal core comprises between 50 to 70 metal atoms, said metal atoms are selected from the group consisting of:
platinum (Pt), gold (Au), ruthenium (Ru), and mixtures thereof;
in which the metal core, including the ligand(s), has an average size of from 1 to 5 nm; and
in which the metal core has an average size of from about 0.5 nm to about 2.5 nm.

45. The metal cluster nanocompound as claimed in claim 37, which is soluble or at least dispersible in aqueous media under physiological conditions due to the selection of suitable ligands.

46. The metal cluster nanocompound as claimed in claim 37, in which the ligand(s) may be organic radicals and/or halogens, selected from the group consisting of the group consisting of: triphenylphosphine, derivatives of triphenylphosphine, halogens, and mixtures thereof.

47. A metal cluster nanocompound as claimed in claim 37 which has the general formula (I)
[MₙLₘ] (I)
where M is a transition metal atom selected from the group consisting of: platinum (Pt), gold (Au), rhodium (Rh), iridium (Ir), palladium (Pd), ruthenium (Ru), osmium (Os), silver (Ag) and mixtures thereof;
n is the number of transition metal atoms per metal cluster nanocompound, with n being at least 30 and no higher than 90;
L is a ligand and may denote identical or different ligands in the same molecule;
m is the number of ligands per molecule and is at least 10; and
physiologically tolerated salts, derivatives, isomers, hydrates, metabolites and/or prodrugs thereof suitable for the prophylactic and/or therapeutic treatment of disorders of the human or animal body.

48. The metal cluster nanocompound as claimed in claim 47, in which M=Au and/or n=55.

49. The metal cluster nanocompound as claimed in claim 47, in which the ligand L is selected from the group consisting of triphenylphosphine, derivatives of triphenylphosphine, halogens; and mixtures thereof.

50. A metal cluster nanocompound as claimed in claim 37 having the general formula (II)
[Au₅₅L′₁₂X₆] (II)
Where L′ denotes identical or different ligands in the same molecule and is selected from the group consisting of: a triphenylphosphine radical, derivatives of triphenylphosphine, P(C₆H₅)₂(C₆H₄SO₃H), and P(C₆H₅)₂(meta-C₆H₄SO₃H);
X is a halogen atom and may denote identical or different halogen atoms in the same molecule;
physiologically tolerated salts, derivatives, isomers, hydrates, metabolites and/or prodrugs thereof; and
suitable for the prophylactic and/or therapeutic (curative) treatment of disorders of the human or animal body.

51. The metal cluster nanocompound as claimed in claim 37, having a water solubility of at least 0.1 μmol/l.

52. The metal cluster nanocompound as claimed in claim 37, formulated for the prophylactic and/or therapeutic treatment of a condition selected from the group consisting of: neoplastic or cancerous disorders of the human or animal body, primary tumors, metastasized tumors, precancerous diseases, colon cancer, colon carcinomas, breast cancers, mamma carcinomas, ovarian carcinomas, carcinomas of the uterus, lung cancer, stomach cancer, liver cancer, carcinomas of the pancreas, kidney cancer, bladder cancer, prostate cancer, testicular cancer, bone cancer, skin cancer, Kaposi sarcomas, brain tumors, myosarcomas, neuroblastomas, lymphomas and leukemias.

53. The metal cluster nanocompound as claimed in claim 37 formulated for the prophylactic and/or therapeutic treatment of benign and malignant tumors.

54. The metal cluster nanocompound as claimed in claim 37, which inhibits and/or stops cell growth and/or cell division of tumor and/or cancer cells and/or which induces destruction of tumor and/or cancer cell DNA.

55. The metal cluster nanocompound as claimed in claim 37, formulated to be administered systemically and/or topically.

56. A pharmaceutical composition or medicament, comprising at least one metal cluster nanocompound as defined in claim 37 and/or physiologically tolerated salts, derivatives, isomers, hydrates, metabolites and/or prodrugs thereof in therapeutically active amounts, together with a pharmaceutically tolerated carrier or excipient.

57. The pharmaceutical composition or medicament as claimed in claim 56, comprising at least one further pharmaceutical active compound, a chemotherapeutic or a cytostatic agent, present either as a mixture or a batch or spatially separated from one another.

58. The pharmaceutical composition or medicament as claimed in claim 56 for systemic and/or topical application.

59. A process for the prevention and/or treatment of disorders of the human or animal body, comprising administering to said human or animal at least one metal cluster nanocompound as defined in claim 37 and/or physiologically tolerated salts, derivatives, isomers, hydrates, metabolites and/or prodrugs thereof in therapeutically active amounts, together with a pharmaceutically tolerated, essentially nontoxic carrier or excipient.

60. The process as claimed in claim 59, in which the metal cluster nanocompound and/or physiologically tolerated salts, derivatives, isomers, hydrates, metabolites and/or prodrugs thereof are administered in combination with at least one further pharmaceutical active compound, present either as a functional unit in the form of a blend a mixture or spatially separated from one another, in which the at least one further pharmaceutical active compound can be administered simultaneously or else sequentially with respect to the metal cluster nanocompounds and/or their physiologically tolerated salts, derivatives, isomers, hydrates, metabolites and/or prodrugs.

61. A metal cluster nanocompound of the formula
[Au₅₅ {P(C₆H₅)₂(C₆H₄SO₃H)}₁₂Cl₆]
and physiologically tolerated salts, derivatives, isomers, hydrates, metabolites and/or prodrugs thereof,
for the prophylactic and/or therapeutic treatment of disorders of the human or animal body.

62. The metal cluster nanocompound as claimed in claim 61 of the formula
[Au₅₅ {P(C₆H₅)₂(meta-C₆H₄SO₃H)}₁₂Cl₆]
and physiologically tolerated salts, derivatives, isomers, hydrates, metabolites and/or prodrugs thereof,
for the prophylactic and/or therapeutic treatment of disorders of the human or animal body.
